(19) United States
(12) Patent Application Publication
(10) Pub. No.: US 2026/0314928 A1
(43) Pub. Date: Oct. 15, 2026

(54) INTRA-OCULAR OPTICAL COHERENCE TOMOGRAPHY APPARATUS AND METHOD FOR VITREORETINAL SURGERY

(71) Applicants: The General Hospital Corporation, Boston, MA (US); Massachusetts Eye and Ear Infirmary, Boston, MA (US)

(72) Inventors: Grace Elizabeth Baldwin, Boston, MA (US); Guillermo Tearney, Boston, MA (US); John Miller, Boston, MA (US); Andres Garcia Coleto, Boston, MA (US); Forrest Nicholas Doherty, Boston, MA (US); Francesco Romano, Boston, MA (US)

(21) Appl. No.: 19/112,009

(22) PCT Filed: Nov. 1, 2023
(86) PCT No.: PCT/US2023/078321

§ 371 Date: Mar. 14, 2025

Related U.S. Application Data
(60) Provisional application No. 63/381,925, filed on Nov. 1, 2022.

(51) Int. Cl. A61B 3/12; A61B 3/10; A61B 5/00; G02B 6/36

(52) U.S. Cl. CPC A61B 3/12; A61B 3/102; A61B 5/0084; G02B 6/3624

(57) ABSTRACT

An optical probe, including; an optical fiber coupled to a moveable actuator, the moveable actuator being configured to direct light emitted from the optical fiber at a plurality of varying angles; and a relay lens distal to the moveable actuator and configured to receive light emitted from the optical fiber and direct the received light at a sample.

### CROSS-REFERENCE TO RELATED APPLICATIONS

The present application is based on and claims priority from U.S. patent application Ser. No. 63/381,925, filed on Nov. 1, 2022, the entire disclosure of which is incorporated herein by reference.

### STATEMENT REGARDING FEDERALLY SPONSORED RESEARCH

N/A.

### BACKGROUND

Optical coherence tomography (OCT) is a noninvasive imaging technology that uses light (e.g., infrared light) to capture high resolution images in tissue. Intra-operative OCT (iOCT), the use of OCT in the operating theater during surgical procedures, serves as a meaningful tool for surgeons in various ophthalmic surgeries. In vitreoretinal surgery, instruments are placed inside the eye to operate on the back inside layer of the eye, where the retina is located. Intraoperative OCT is particularly useful during retinal surgery as it allows delineation of the individual layers of the retina which aids in surgical decision making. Current intraoperative OCT devices are all extraocular, which limits the ability to image certain structures and guide surgical movements. Intra-ocular OCT, on the other hand, would involve inserting a OCT probe directly into the eye. There are no clinically approved intraocular OCT devices and few device designs exist. Among the small number of prior designs, they all appear to lack design criteria for a device that is robust, cost-effective, and scale-able, thereby limiting the clinical feasibility.

### SUMMARY OF THE INVENTION

Accordingly, new systems, methods, and apparatus for resolving a clinically feasible, cost-effective, and scale-able intraocular optical coherence tomography device for vitreoretinal surgery are desirable.

The development of OCT has revolutionized the practice of ophthalmology including our understanding and clinical management of numerous ophthalmic diseases.

These advances subsequently led to the introduction of OCT in intra-operative settings, which appears to be a meaningful tool for various ophthalmic surgeries, in particular vitreoretinal surgery, since OCT enables delineation of individual retinal layers and pathologies within the retina. The integration of this technology within the instrument (intraocular OCT) would possess certain advantages over existing intra-operative OCT devices, which image the retina from outside the eye. In particular, intra-ocular OCT would allow for guidance of surgical procedures, may better aid in surgical decision making by improving intra-operative diagnosis, and pave the way for future technology and advances within the field.

In conventional intra-operative OCT, the extraocular images cause the surgical instruments to create shadowing which inhibits the ability for surgical movements to be guided. To obtain a quality image the instruments often must be removed and then re-inserted. An intra-ocular OCT probe, on the other hand, would enable direct imaging that would happen in concert with the surgical instruments already inserted in the eye. Therefore, guidance of surgical procedures, particularly submacular surgeries such as gene therapy, would be greatly improved through intra-ocular OCT.

In addition, any media opacity, such as corneal edema, dense cataract, or other anterior segment disease, as well as intraocular opacity, such as vitreous hemorrhage or dense vitritis obscure the imaging acquired with conventional, extraocular OCT. With an intra-ocular OCT probe, it would directly image the retina and the image would not be affected by media or intraocular opacity.

Intraocular OCT, compared to intra-operative OCT, would likely allow for better visualization of peripheral structures within the retina, since the probe could be adjusted to image the far periphery. This may enable to the visualization of subclinical breaks in the retina, that otherwise would have been missed, and may improve outcomes in retinal detachment surgery.

In the case of epiretinal membrane peeling, often dyes are used to mark the borders of the membrane. Intraocular OCT would enable direct visualization of the epiretinal membrane which would enable adequate peeling and may limit the requirement of dyes, which sometimes have toxic effects.

Overall, the precision of a vast array of surgical procedures within vitreoretinal surgery, and other ophthalmic fields and microsurgical fields, would likely be improved by having a small enough, linear scanning OCT probe that could be inserted directly into the operating field.

Few intraocular OCT devices have been developed to date, all of which appear to lack design criteria that would allow for a clinically feasible, cost-effective, and a scale-able device. Thus, we disclose herein embodiments of a novel intraocular device that has advantages over prior devices and may pave the way for intraocular OCT being utilized in vitreoretinal surgery and other ophthalmic surgeries. The disclosed device has the potential to impact the field of vitreoretinal surgery and improve patient outcomes from both common and uncommon vitreoretinal surgeries. Further, the described device may be used in other fields of ophthalmic surgery, such as anterior segment, oculoplastics, and neuro-ophthalmology to name a few, as well as other surgical fields, such as neurosurgery and otorhinolaryngology, and any other surgical field that utilizes microsurgical techniques. Further, any surgical or medical field that may benefit from high resolution imaging with a forward viewing, linear scanning, OCT probe would likely benefit, such as the surgical resection of tumors, among other applications.

Compared to known intra-ocular OCT probes, the disclosed device contains a novel optical design and a novel mechanical design. To the inventors' knowledge, no prior intra-ocular OCT devices have used a micro electromechanical (MEMS) microscanning mirror system. This system is scale-able compared to prior actuators and allows for 3D volumetric imaging, which is not possible with prior actuator systems reported for intraocular OCT.

Also disclosed is a novel mechanical design, which optimizes the device to be clinically feasible, reusable, cost-effective, and scale-able. An important aspect of the design is that the distal cap which contains the optical elements inserted into the eye would be detachable and thus a single-use item, while the remaining components proximal to this would be reusable. This would allow the expensive regions of the device (actuator, fiber collimator, and electrical components) to be used repeatedly in the clinical setting, while still enabling high level of sanitation and safety as the intraocular components would be one-time use. A one-time use cap would significantly reduce the likelihood of causing infections in the eye or transmitting infections, such as prion diseases, from one patient's eye to the other.

Another novel aspect of this design is that collimated light is relayed from the handpiece all the way though the probe tip. To the inventors' knowledge, none of the known intra-ocular OCT devices translate collimated light to the probe tip and instead use a single mode fiber that passes from the handpiece into the probe to deliver light. Our collimated light relay design makes the probe more clinically feasible, durable, and able to be used repeatedly.

The mechanical design that centers the optical elements including the fiber collimator, MEMS, GRIN lens, and other optics has been designed specifically for the device. The optics (on the distal end of the device) may include a long relay lens which may be based on a GRIN lens design, which known intra-ocular OCT devices have not described.

In addition to the above-described aspects, the device may include one or more of the following features. In some embodiments, the device may include a relay lens which includes stacked GRIN lenses, multiple GRIN lenses at a distal end, and/or a custom single GRIN lens at the distal end. In other embodiments, the device may include a relay lens which includes a single-mode (SM) fiber extending through a support structure (e.g., a hypotube or needle), a ball lens at a distal end as the optics, and/or a multimode fiber as the optics. In still other embodiments, the device may include a relay lens which includes a multimode GRIN fiber in place of the relay lens.

Possible actuator designs, in addition to the disclosed MEMS-based scanning mechanism, include: a rotary motor and/or miniature galvanometer device; a piezoelectric actuator; use of electromagnetics and/or a shape memory alloy; electrostatics; or electrothermal mechanisms.

Other improvements provided by embodiments of the disclosed device include increasing the field of view with an umbrella design on the distal tip; increasing the field of a view with a balloon at the end that may be filled with saline or another substance, such as ferromagnetic fluid, silicone, air, or gas; using dye to mark locations on the tissue (retina or other tissues) guided by the OCT device intraoperatively; simultaneous white, blue, or other spectrum light imaging in addition to OCT imaging; use of a curved probe or angled probe; optimization of the probe and OCT technology for imaging through one or more of air, oil, or gas; use of a probe that is configured to safely make contact with the retinal tissue in a manner that does not damage the retina and thereby helps to stabilize the probe while imaging; use of a probe in which lasering of tissue is performed through the same probe as is used for imaging; a probe that is optimized for other forms of ophthalmic surgery by modifications to optics, including but not limited to: corneal surgery, oculoplastic surgery, neuro-ophthalmology, strabismus surgery, pediatric surgery, and/or glaucoma surgery, or other surgical fields, such as neurosurgery or otorhinolaryngology, or any surgery that utilizes microsurgical techniques or requires high resolution tissue imaging, such as cancer resections and monitoring. Each of these applications within other fields of ophthalmology and other surgical or medical fields is expected to be a straightforward application of the described technology. In some cases, the exact same probe with identical specifications could be utilized. In other cases, minor modifications, for example adjusting the working distance or increasing field of view through modification of the optics (GRIN lens), likely at the cost of diameter and resolution, would be modifications to the existing design that would enable devices well-suited for other surgical or medical procedures.

In various embodiments, prior described applications of intra-ocular OCT that would be feasible with this design include simultaneous surgical procedures that could be guided and aided by the device, including one or more of: use of micro-forceps, lasering, injections, cryotherapy, biopsy, suction, delamination and segmentation, posterior vitreous detachment induction, dye injection, or cutting.

Some embodiments provide for device optimization for imaging pediatric patients and for use in other fields, such as anterior segment surgery, and other fields of ophthalmology. Still other embodiments provide integration with heads up visualization devices, motion compensation, surgical step guidance, smart detection and instrument tracking and artificial intelligence, integrated working port or blade, and/or ability to insert the probe into a working channel of another device.

Various embodiments provide an optical probe and a method of operating an optical probe. The optical probe includes: an optical fiber coupled to a moveable actuator, where the moveable actuator may be configured to direct light emitted from the optical fiber at a plurality of varying angles; and a relay lens distal to the moveable actuator and configured to receive light emitted from the optical fiber and direct the received light at a sample. The method includes: providing an optical fiber coupled to a moveable actuator; directing light emitted from the optical fiber at a plurality of varying angles; receiving, by a relay lens distal to the moveable actuator, light emitted from the optical fiber; and directing the received light at a sample.

In some embodiments, the plurality of varying angles may include angles of up to 3.75 degrees on either side of an optical axis. In other embodiments, the plurality of varying angles may include angles of up to 5 or 10 degrees on either side of an optical axis. The optical axis can scan at varying angles depending on the design and MEMS mirror used. For example, MEMS mirrors range in size typically from 2-7.5 mm, among other sizes, and having varying degrees of angles, often from −7 to +7 degrees. Depending on the size of the probe desired, a different MEMS mirrors can be used. The angle at which the MEMS mirror bends the light is related to the size of the probe and the desired field of view which is optimized according to optical modeling. For example, for a 25-gauge probe, with a 1 mm FOV, our optical modeling found that a MEMS mirror scanning at 0.8 degrees on either side of the optical axis at the end of the probe scanning 5 degrees on either side. Beyond MEMS mirrors, other actuators may be used as well as described herein, such as electromagnetic, micromotor, piezoelectric, among others, which may result in other varying angles that could be scanned, again respective of the size of probe, desired field of view, and clinical application.

In various embodiments, the optical probe may further include a fiber collimator disposed at a distal end of the optical fiber; a fixed reflective surface disposed distal to the fiber collimator; and an adjustable reflective surface coupled to the moveable actuator and disposed adjacent the fixed reflective surface. The adjustable reflective surface may be configured to reflect light emitted from the fiber collimator and reflected off the fixed reflective surface at the plurality of varying angles. The relay lens may be configured to receive light reflected from the adjustable reflective surface and direct the received light at the sample.

In certain embodiments, the moveable actuator may include at least one of a Microelectrical Mechanical System (MEMS) device, a galvanometer, or a rotary motor.

In some embodiments of the optical probe, the relay lens may be disposed within a distal cap and the distal cap and the relay lens may be detachable from the optical probe.

In various embodiments of the optical probe, the relay lens may include an objective lens at a distal end thereof. In some embodiments of the optical probe, the relay lens may include a GRIN lens. In particular embodiments of the optical probe, the GRIN lens may include a plurality of stacked GRIN lenses. In certain embodiments of the optical probe, the relay lens may be a curved or angled probe and the relay lens may include at least one of an angle-polished GRIN lens or a prism that is configured to direct light through the curved or angled probe.

In some embodiments, the optical probe may further include a telecentric lens disposed between the moveable actuator and the relay lens, where the telecentric lens may be configured to focus the light emitted from the optical fiber onto a proximal end of the relay lens.

In various embodiments of the optical probe, the moveable actuator may rotate on a first axis such that the received light is scanned in a first direction across a surface of the sample. In other embodiments of the optical probe, the moveable actuator may rotate on a second axis perpendicular to the first axis such that the received light is scanned in a second direction perpendicular to the first direction across the surface of the sample.

In various embodiments of the optical probe, the fixed reflective surface may include at least one of a mirror or a prism.

In particular embodiments of the optical probe, the optical fiber and the moveable actuator may be disposed within a hand piece. In some embodiments of the optical probe, the hand piece may include a lower portion and an upper portion, and the optical fiber and the moveable actuator may be disposed in the lower portion of the hand piece to facilitate optical alignment and the upper portion of the hand piece may be secured to the lower portion of the hand piece. In certain embodiments of the optical probe, the hand piece may further include a plurality of adjustment mechanisms associated with at least one of the optical fiber and the moveable actuator, where the plurality of adjustment mechanisms may be configured to facilitate the optical alignment. In particular embodiments of the optical probe, the hand piece may be disposed within an outer housing.

In some embodiments of the optical probe, the fiber collimator, the fixed reflective surface, the moveable actuator, and the adjustable reflective surface may be disposed within a hand piece. In various embodiments of the optical probe, the hand piece may include a lower portion and an upper portion, and the fiber collimator, the fixed reflective surface, the moveable actuator, and the adjustable reflective surface may be disposed in the lower portion of the hand piece to facilitate optical alignment and the upper portion of the hand piece may be secured to the lower portion of the hand piece. In certain embodiments of the optical probe, the hand piece may further include a plurality of adjustment mechanisms associated with at least one of the fiber collimator, the fixed reflective surface, the moveable actuator, and the adjustable reflective surface, where the plurality of adjustment mechanisms may be configured to facilitate the optical alignment. In particular embodiments of the optical probe, the hand piece may be disposed within an outer housing.

In various embodiments of the optical probe, the relay lens may be disposed within a hypotube. In some embodiments of the optical probe, the hypotube may include a plurality of channels and the relay lens may be disposed within a first channel of the plurality of channels. In certain embodiments of the optical probe, the plurality of channels may include at least two channels separated by a septum. In particular embodiments of the optical probe, the plurality of channels may include a plurality of concentric channels, where the relay lens may be disposed within a first central channel. In some embodiments of the optical probe, a second outer channel may include at least one of a cutting blade, a suction port, an air delivery port, a fluid delivery port, or a medicament delivery port.

In certain embodiments, the optical probe may further include an optical coherence tomography (OCT) system coupled to the optical fiber. In various embodiments of the optical probe, the OCT system may further include an OCT light source coupled to the optical fiber. Particular embodiments of the optical probe may further include a white light source coupled to the optical fiber.

In various embodiments of the optical probe, the sample may include a retina.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1, panel A shows a photograph demonstrating instruments being inserted to an eye undergoing vitreoretinal surgery.

FIG. 1, panel B illustrates how instruments are inserted into the eye to allow for surgical procedures to be performed on the retina, located at the back of the eye, including a light source and an intraocular optical coherence tomography (OCT) probe.

FIG. 2 shows how the intraocular OCT probe of FIG. 1, panel B would be utilized during a vitreoretinal surgery, including a monitor including a light probe image and an OCT image, an OCT probe, a swept source OCT system, a power source for an actuator, and a light pipe.

FIG. 3, left panel, shows a photograph demonstrating how a video endoscope is utilized in vitreoretinal surgery, including a light pipe image of a retina surface, an endoscope image, a split screen view, a microscope, an intra-ocular video endoscope, and an optical fiber transmitting white light.

FIG. 3, right panel, shows a photograph demonstrating how an OCT probe is utilized in vitreoretinal surgery, including an OCT image, a light pipe image of a retina surface, a split screen view, a microscope, an intra-ocular OCT probe, and an optical fiber transmitting 1060 nm light.

FIG. 4, panel A illustrates an optical design and specification for the intraocular OCT probe of FIG. 1B, including a relay lens and an objective lens.

FIG. 4, panel B illustrates potential optical designs for the intraocular OCT probe of FIG. 1B, including a custom GRIN lens (1); a design using 3 off-the-shelf GRIN lenses (2); a single mode (SM) fiber or collimated light combined with a plurality of stacked off-the-shelf GRIN lenses and a polished off-the-shelf GRIN lens (3); and a single relay lens combined with an objective lens (4).

FIG. 4, panel C illustrates a diagram of hand-piece optics and a laser in an eye, the handpiece including a scan mirror.

FIG. 5, panel A shows an illustration of an intraocular OCT device design using a Microelectrical Mechanical System (MEMS) scanning system, showing that the distal cap and the relay lens are one-time use components.

FIG. 5, panel B shows an illustration of an intraocular OCT device design with insets highlighting a fiber collimator and a MEMS device.

FIG. 6, panel A shows a lateral three-dimensional rendering of a mechanical design of an intraocular OCT probe.

FIG. 6, panel B shows a cross-sectional three-dimensional rendering of an intraocular OCT probe, showing placement of a fiber collimator, a prism, and a MEMS mount.

FIG. 6, panel C shows a cross-sectional three-dimensional rendering of an intraocular OCT probe, including a strain relief, a proximal cap, a fiber collimator, a prism, and a MEMS mirror.

FIG. 7, panel A illustrates the lower half of the body of an intraocular OCT probe demonstrating a V-groove for optical alignment of a fiber collimator, a MEMS holder, set screws, and a holder for GRIN lens and a telecentric lens.

FIG. 7, panel B illustrates the lower half of the body of an intraocular OCT probe demonstrating a fiber collimator directing a beam of light into a fixed reflector (right angle mirror) where the beam then reflects off a MEMS mirror and into a GRIN lens disposed in a hypotube.

FIG. 7, panel C illustrates the lower half of the body of an intraocular OCT probe demonstrating a close-up image of a beam emitted from a fiber collimator reflecting off a fixed reflector (a flat mirror), and then off a moveable reflector (a MEMS mirror) where it would be directed to a relay lens and toward a sample.

FIG. 8 illustrates the alignment of optical components on the interior of an intraocular OCT probe, showing how the beam from the collimator reflects from a right angle mirror towards a MEMS mirror, through a telecentric lens, and into a hypotube containing a relay lens.

FIG. 9, panel A shows side perspective (left panel) and front (right panel) views of a telecentric lens holder with three points of contact for optimal positioning of the telecentric lens within the lens holder.

FIG. 9, panel B shows front (left panel) and rear (right panel) views of a telecentric lens holder with three points of contact for optical positioning that show an insertion opening on the rear face (right panel) for a hypotube to indicate how the hypotube and telecentric lens holder are associated with one another.

FIG. 10, panel A shows a lateral perspective view of the assembled lower and upper housing pieces of the body of an intraocular OCT probe, highlighting the upper “clam shell” component.

FIG. 10, panel B shows a distal cutaway view of the body of an intraocular OCT probe, including a lower clam shell, an upper clam shell, and an outer cosmetic hand-piece that may be placed over the assembled clam shell components.

FIG. 11, panel A shows a design of an outer “cosmetic” component of an intraocular OCT probe as in FIG. 10, panel B, which transitions from the probe body/hand-piece to a hypotube with an intervening strain relief using a solid tube design.

FIG. 11, panel B shows another design of an outer “cosmetic” component of an intraocular OCT probe as in FIG. 10, panel B, which transitions from the probe body/hand-piece to a hypotube with an intervening strain relief using an ergonomic handle design.

FIG. 12, panel A shows optical modeling of an entire intraocular OCT probe.

FIG. 12, panel B shows optical modeling of a distal tip of an intraocular OCT probe.

FIG. 12, panel C shows optical modeling of a relay lens with a plurality of stacked GRIN lenses design of an intraocular OCT probe.

FIG. 13 shows optical modeling (left panel) evaluating a spot size and field of view (FOV) for a beam at the center of the FOV (0 mm) and at the edge of the FOV (+0.5 mm) using a probe including a fiber collimator, a flat mirror, a MEMS mirror, and a GRIN lens.

FIG. 14, panel A (option 1), shows optical modeling of a probe having a single GRIN rod lens with an outer diameter of 0.35 millimeters.

FIG. 14, panel B (option 2), shows optical modeling of a probe having a single GRIN rod lens with a telecentric coupling lens.

FIG. 14, panel C (option 3), shows optical modeling of a probe which includes a relay lens which includes a relay GRIN lens plus an objective GRIN lens.

FIG. 15, panel A (option 1), shows optical modeling of a proximal optical system with a single GRIN rod lens of the probe of FIG. 14, panel A (option 1).

FIG. 15, panel B (option 2), shows optical modeling of a proximal optical system with a single GRIN rod lens with a telecentric coupling lens of FIG. 14, panel B (option 2).

FIG. 16 shows optical modeling of an output spot size with a probe having a single relay lens and no objective lens; the inset shows a through-focus series of spots at different focal levels at a point off the central axis (top row) or on the axis (bottom row).

FIGS. 17A and 17B show data pertaining to a tolerancing analysis for each device component of an intraocular OCT probe.

FIG. 18 shows data pertaining to throughput and back reflection analyses for a system of an intraocular OCT probe, indicating that total one-way transmission is almost 85% and total back reflection collection is 0.0275%.

FIG. 19, panel A, shows an intraocular OCT device design using a MEMS scanning system as the moveable actuator.

FIG. 19, panel B, shows an intraocular OCT device design using a single fiber actuator as the moveable actuator.

FIG. 19, panel C, shows an intraocular OCT device design using a rotary motor or galvanometer scanning system as the moveable actuator.

FIG. 20 shows an intraocular OCT device design that accommodates a rotary motor that extends beyond the surface of the housing through modifying the hand-piece design of the of the intraocular OCT probe; the top panel has the distal cap and hypotube/relay lens attached and in the bottom panel the distal cap and hypotube/relay lens have been separated from the body of the probe.

FIG. 21 shows a curved or angled probe design that can be used for imaging a peripheral retina, as shown in the left panel; the right panel shows a component such as an angle-polished GRIN lens or prism that can be included in the relay lens to direct light in a curved or angled pathway in the curved or angled probe design.

FIG. 22 shows a probe design for combining both white light imaging and OCT imaging simultaneously, including a white light source combined with an infrared light source that is coupled to an OCT probe. The top panel shows a basic system schematic and the bottom panel includes insets showing insertion of a probe into the eye and example white light and OCT images.

FIG. 23, panel A, shows a design of a multi-channel intraocular OCT probe with an OCT lens or fiber in the center channel and one or more of suction, air, injected fluid, or medicament being applied to one or more outer channels.

FIG. 23, panel B, shows a design of a multi-channel intraocular OCT probe with an OCT lens or fiber in the center channel and a biopsy or cutting blade disposed in one or more outer channels.

FIG. 23, panel C, shows a design of a multi-channel intraocular OCT probe with a septum between the channels and an OCT lens or fiber in one of the channels and a second channel being available as a working port.

### DETAILED DESCRIPTION

In accordance with some embodiments of the disclosed subject matter, mechanisms (which can include systems, methods, and apparatus) for resolving a clinically feasible, cost-effective, and scale-able intraocular optical coherence tomography device for vitreoretinal surgery are provided.

The development of optical coherence tomography (OCT) has revolutionized the practice of ophthalmology including our understanding and clinical management of numerous ophthalmic diseases. Intraocular OCT possess certain advantages over current intra-operative OCT devices, and allow for guidance of surgical procedures, particularly for submacular surgeries including gene therapy.

Optical coherence tomography (OCT) began as a research project collaboration between Massachusetts Eye and Ear, Harvard Medical School, and Massachusetts Institute of Technology in the 1980s. Since then, OCT has revolutionized the field of ophthalmology. Today, OCT is standard of care for the management of various ophthalmic diseases, and in particular retinal diseases.

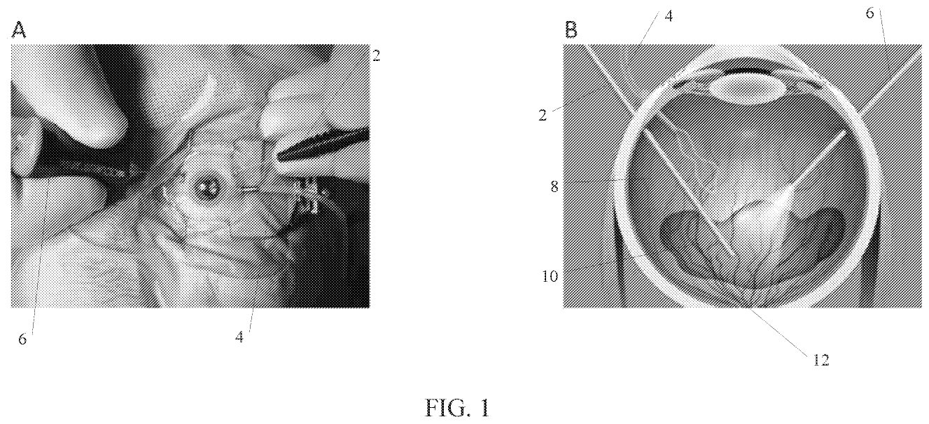

Intra-operative OCT (iOCT), the use of OCT in the operating theater during surgical procedures, was first implemented in 2005 by coupling a beam splitter to the front of a surgical microscope. To date, various iOCT devices exist, including hand-held OCT and microscope-integrated OCT, and are routinely used in numerous types of ophthalmic surgeries. Evidence derived from clinical trials suggests that iOCT benefits vitreoretinal surgery, as exemplified by the DISCOVER trial that found that iOCT altered decision making in 29% of posterior segment surgeries and the PIONEER trial that found that iOCT altered decision making in 46% of membrane peeling cases-a common vitreoretinal surgery. These studies have demonstrated the utility of iOCT to impact vitreoretinal surgery, however there are important limitations of current iOCT devices. During vitreoretinal surgery, instruments are placed inside the eye (vitreous cavity) to operate on the tissue of interest, the retina in this case (FIG. 1). Since current iOCT devices are working external to the eye, surgical instruments may create artifacts and thus impair accurate imaging, making the coordination between instrument movement and OCT visualization complicated. In addition, application of these procedures to patients with media opacities, such as clouding of the cornea or dense cataracts, remains challenging. Further, visualizing the far periphery of the retina by means of microscope-integrated iOCT is not feasible due to optical aberrations and the wide focus differences. In this regard, clinical trials suggest that it is in complex cases that iOCT provides the most value for the surgeon, and it is these same complex cases that are more likely to have media opacity and require imaging of the retinal periphery.

An endoscopic OCT probe that is directly inserted into the eye would limit the aforementioned challenges of external iOCT. Further, an intraocular endoscopic OCT probe would have the added ability to directly guide surgical maneuvers. There are numerous potential clinical applications for intraocular OCT, which spans from routine vitreoretinal procedures to recent and future advances of the field, such as gene therapy. During pars plana vitrectomy (PPV), a procedure performed on nearly every patient a vitreoretinal surgeon operates on, an intraocular OCT probe could be used to verify complete posterior vitreous detachment and removal of peripheral vitreous, helping to prevent post-operative complications. Today, epiretinal membrane (ERM) peeling is directed by fluorescent dyes that may exert a toxic effect if trapped underneath the retina; intraocular OCT may circumvent the need for any dyes and assure complete removal of the ERM by directly visualizing the most feasible location to begin peeling and the presence of remaining epiretinal tissue. During retinal detachment repair, intraocular OCT could be used to identify any remaining subretinal fluid, breaks, residual perfluorocarbon liquid, and initial proliferative vitreoretinopathy, as well as helping to differentiate schisis from retinal detachment. Intraocular OCT could aid in macular hole surgeries, identifying vitreomacular traction, fibrous tissues during endophthalmitis cases, and be used during subretinal and choroid biopsies. Submacular surgeries is an exciting field that intraocular surgery may aid in the surgeries for the novel technologies in this area, including gene therapy (example: AAV2-REP1 for choroideremia), retinal prosthesis implantation (example: Argus II), and subretinal tissue plasminogen activator injection for submacular hemorrhage. In particular, optimized induction of bleb formation during gene augmentation therapy is becoming critical due to the recent description of perifoveal chorioretinal atrophy after subretinal injection of voretigene neparvovec-rzyl, which might be in part due to the injection technique.

However, intraocular OCT applications are not limited to the posterior segment. In anterior segment surgery, intraocular OCT may help in glaucoma surgery during ab externo and ab interno procedures in order to better visualize trabecular structures and uveo-scleral pathways. During corneal transplantation, intraocular OCT could aid in adherence of posterior lamellar keratoplasty, bypassing the air-tissue interface and especially in the presence of subepithelial and anterior stromal opacities. Intraocular OCT may also help in complex cataract surgeries and various pediatric ophthalmic surgeries.

A small number of intraocular OCT devices have been described. Nearly all were tested in ex vivo animal tissue, as opposed to in an in vivo setting, and only two devices were used in multiple human patients. One of the devices was not optimized for retinal imaging, as the device was a commercially-available endovascular OCT probe that acquired images in a circular format. In another study, the feasibility of an iOCT-based sensor in assisting robotic vitreoretinal surgery was evaluated. The device was tested on five vitreoretinal cases and was able to perform almost every pre-determined surgical task in their series—including insertion and movement of instruments and testing of a virtual bound. Nevertheless, further improvements are desirable.

Accordingly, disclosed here are embodiments of a novel intraocular OCT device optimized for vitreoretinal surgery. Embodiments of the disclosed device design overcome certain limitations of prior devices, suggesting it has potential for large-scale manufacturing and clinical feasibility.

FIG. 1A shows a photograph demonstrating instruments being inserted to an eye undergoing vitreoretinal surgery. The vitreoretinal surgery may include a vitrector 2 or a cannula 4 that is used for maintaining a closed chamber, reducing infusion fluid, and maintaining the intraocular pressure during surgery. Further included during surgery is a light probe 6 configured to illuminate the retina and vitreous cavity. The vitreoretinal surgery additionally may include an intraocular optical coherence tomography (OCT) probe to ensure precise anatomic subretinal delivery and provide immediate feedback to surgeons following surgical manipulations during surgery.

FIG. 1B is a diagram which illustrates how instruments are inserted into the eye to facilitate surgical procedures performed on the retina 12 (located at the back of the eye, with blood vessels 10 and vitreous humor 8 being indicated), including a light probe 6 and, according to embodiments disclosed herein, may further include an intraocular optical coherence tomography (OCT) probe. In some embodiments, the OCT probe may be configured to allow for three-dimensional volumetric imaging.

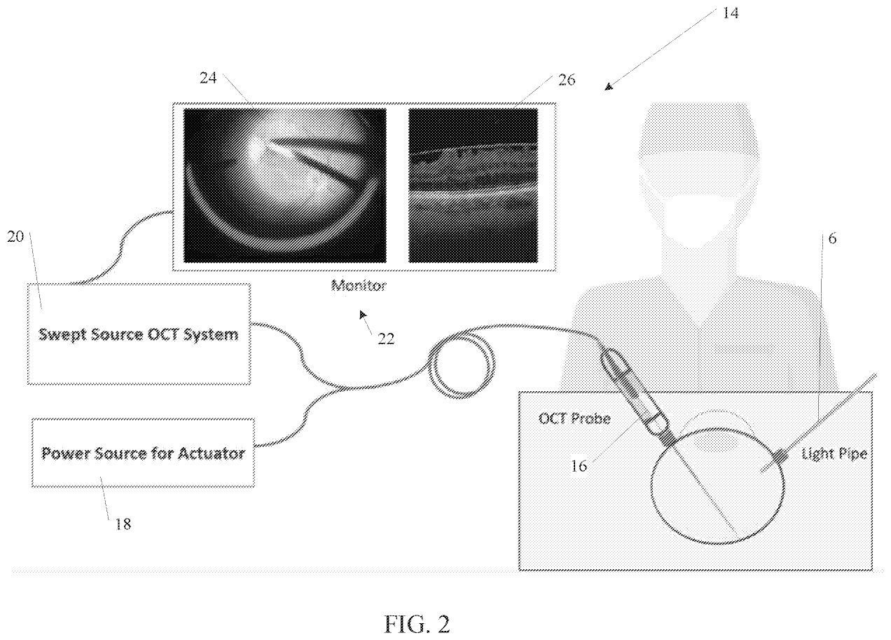

During surgery, the intraocular OCT probe would be inserted into a 25-gauge, or similar, vitrectomy port, as is routinely used for insertion of other vitreoretinal instruments (FIG. 2). The probe would be connected to an OCT system and a power source and then the OCT images would be projected onto a split screen monitor or external monitor (FIG. 2).

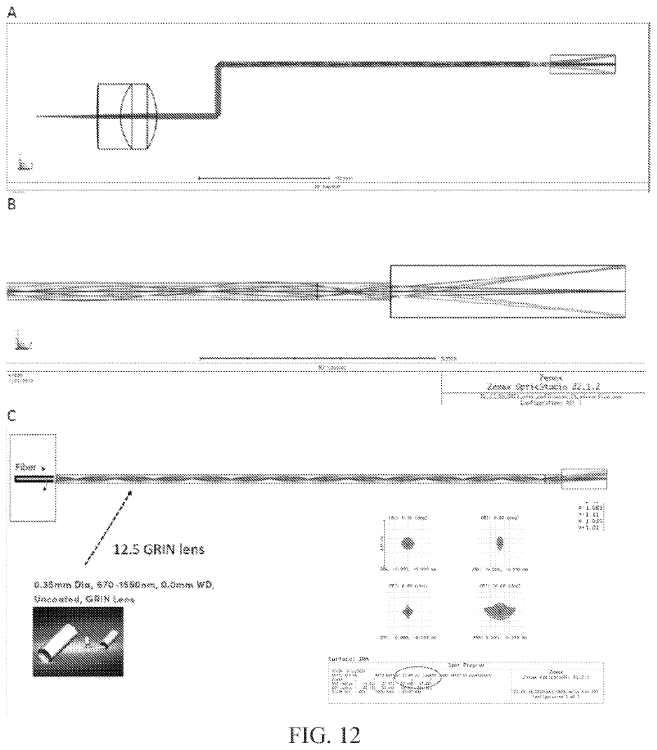

Varying types of OCT systems exist and would be feasible with the device, including time-domain, spectral-domain, and swept source. OCT systems in ophthalmology have advanced, thus many are now spectral domain or swept source-based systems. A swept source OCT system is preferred due to established advantages of SS-OCT over SD-OCT, including higher sensitivity, lower signal to noise, and increased scanning speeds which lead to higher quality OCT images. In addition, the commonly available central wavelength of SS-OCT, 1060nm, penetrates water with less absorption than the typical wavelengths used by SD-OCT (see optical modeling in FIGS. 12-16). FIG. 2 shows an embodiment of an intraocular OCT probe system 14 to be utilized during a vitreoretinal surgery, the system 14 including a monitor 22, a light probe/pipe 6, a swept source OCT system 20, and a power source for an actuator 18. During surgery, a distal portion of the intraocular OCT probe 16 (e.g., the relay lens) may be inserted into the eye via a hypotube or 25-gauge (or similar) vitrectomy port. The OCT probe 16 is connected to the swept source OCT system 20 and the power source 18. This allows OCT images to be obtained and presented on a split screen monitor at the operating site and/or on an external monitor. The split screen monitor is configured to display a white light image 24 of a retina surface and/or an OCT image 26.

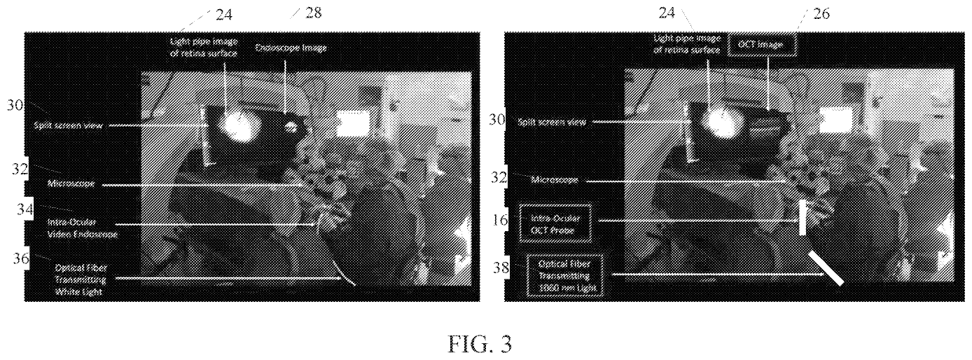

The procedures for using the disclosed intraocular OCT probe are comparable to existing procedures for using endoscopic video imaging probes for vitreoretinal surgery, although instead of or in addition to transmitting white light, the fiber and light probe would transmit white and/or infrared light and the images would include high-resolution OCT and/or white light images (FIG. 3). The left and right panels of FIG. 3 contrast the differences between the use of a conventional video endoscope (left) and an embodiment of the disclosed intraocular OCT probe (right) in vitreoretinal surgery. The left panel of FIG. 3 shows a photograph demonstrating how a video endoscope is utilized in vitreoretinal surgery, including a split screen view 30 that includes a light pipe image 24 of a retina surface and an endoscope image 28. The surgical setup further includes a microscope 32, an intra-ocular video endoscope 34, and an optical fiber transmitting white light 36.

The right panel of FIG. 3 shows a photograph demonstrating how an OCT probe may be utilized in vitreoretinal surgery, which would also include a split screen view 30 that includes a high-resolution OCT image 26 and a light pipe image 24 of a retina surface. Further included would be a microscope 32, an intra-ocular OCT probe 16, and an optical fiber transmitting infrared (e.g., 1060 nm) light 38; in various embodiments, the infrared light for OCT may be combined with white light in order to obtain white light images along with the OCT images. In comparison with using a video endoscope during vitreoretinal surgery, the endoscopic image is replaced with the high-resolution OCT image, the intra-ocular video endoscope is replaced with the intra-ocular OCT probe, and the optical fiber transmitting white light is replaced with the optical fiber that transmits 1060 nm light.

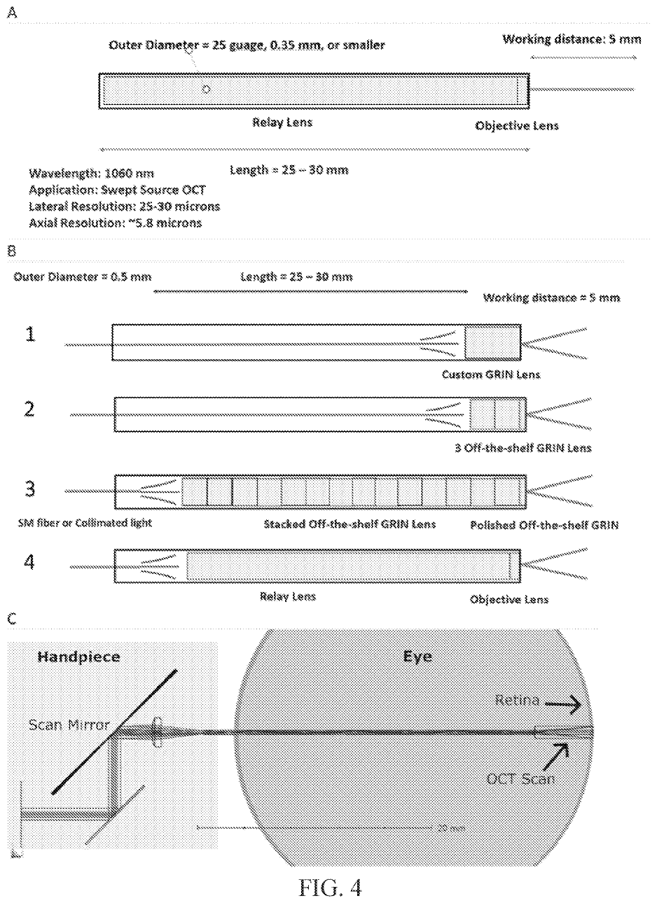

The specifications and safety concerns for the device have been carefully considered after reviewing prior and existing OCT devices, clinical applications and feasibility and ANSI Z136.1 standards for laser safety. In various embodiments, the outer diameter of the probe tip (e.g., the relay lens) will fit inside of a 25-gauge port (0.51 mm) or similar hypotube, or another size port such as a 23-gauge port, or smaller or larger sizes depending on the specific procedure, and the length of the probe tip will be in the range of 25-30 mm given the axial length of the eye (FIG. 4). The targeted lateral resolution is 25 microns, and the optical axial resolution will be equal to or lower than 8.3 microns. The ranging depth will be 1.765 mm, penetration depth 0.90 mm, and the field of view of at least 1 mm. The working distance will be approximately 5 mm to assure no physical or radiation-induced damage to the retina occurs during the procedure while maximizing image quality. The scan type will be linear as is conventional for imaging the retina. Imaging will likely be performed at 1060 nm light as is typically used for swept-source OCT retina imaging, however other light sources are possible, such as 1310 nm light.

FIG. 4A illustrates an optical design and specification for the intraocular OCT probe, including a relay lens and an objective lens. The relay lens is configured to copy the retinal image from the proximal to the distal end of the OCT probe, whereas the objective lens focuses the light at a desired working distance. FIG. 4B illustrates potential optical designs for the intraocular OCT probe of FIG. 1B, including a custom GRIN lens (1); a design using 3 off-the-shelf GRIN lenses (2); an SM fiber or collimated light combined with a plurality of stacked off-the-shelf GRIN lenses and a polished off-the-shelf GRIN lens (3); and a single relay lens combined with an objective lens (4). FIG. 4C illustrates a diagram of hand-piece optics and a laser in an eye. The handpiece includes a fiber collimator, and a scan mirror. The eye includes a retina and an OCT scan.

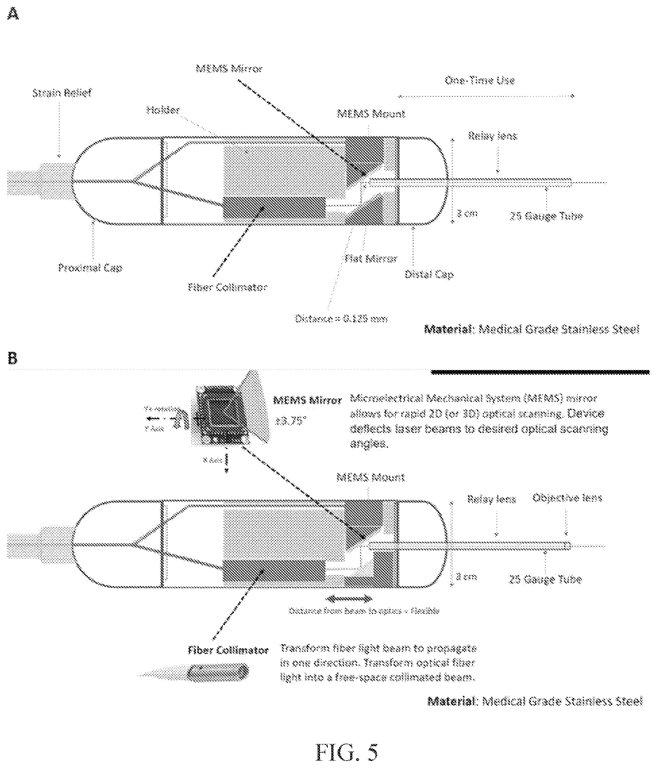

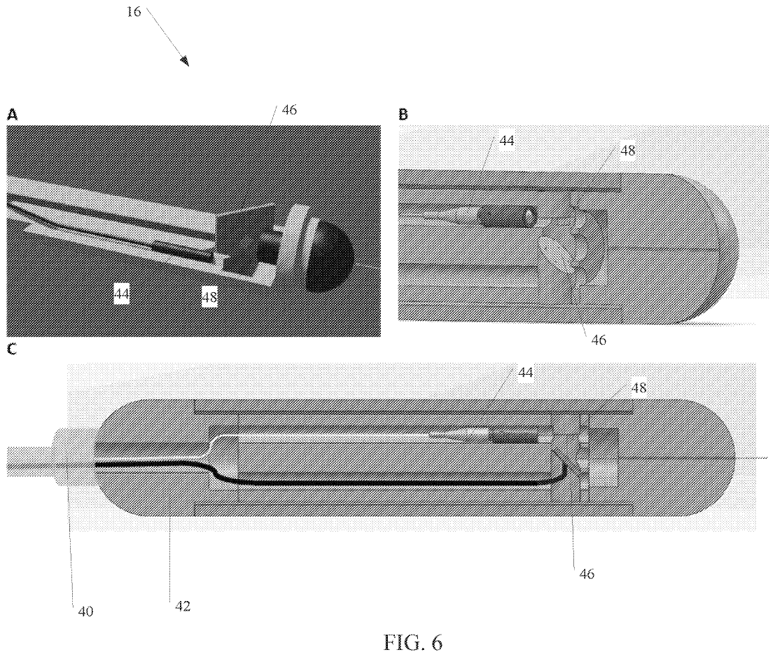

The overall device itself will include two primary components: (1) a re-usable hand-piece containing components that can easily withstand sterilization and (2) a one-time use tip containing the distal cap as well as the focusing and relay optics (FIGS. 5 and 6). In the reusable hand-piece, light from the OCT system will travel through an optical fiber and then be transformed by a fiber collimator to propagate in one direction as a free-space collimated beam (FIGS. 5A, 5B). This light will be reflected by a stationary reflector (e.g., a mirror or prism) to movable reflector such as a Microelectrical Mechanical System (MEMS) mirror (FIGS. 5A, 5B). MEMS-controlled mirrors are particularly well suited as they allow for rapid 2D or 3D optical scanning by deflecting laser beams at a desired optical scanning angle. The MEMS mirror will direct and scan the light into the optics, eventually reaching the retinal tissue (FIGS. 5A, 5B). In various embodiments, the MEMS mirror is the final optic in the reusable hand-piece side.

FIGS. 5A and 5B provide mechanical illustrations of embodiments of the intraocular OCT device design, specifically using a MEMS scanning system to move the moveable reflector. In other embodiments, a single fiber actuator and/or a rotary motor or galvanometer scanning system may be used to move the moveable reflector. FIG. 5A shows a mechanical illustration of an intraocular OCT device design using a MEMS scanning system, indicating a strain relief, a proximal cap, a holder, a fiber collimator, a MEMS mount that includes a MEMS mirror, and a fixed or stationary flat mirror. The Microelectrical Mechanical System (MEMS) mirror allows for rapid two-dimensional or three-dimensional optical scanning. The MEMS mirror is configured to deflect the laser beams into a desired optical scanning angle within a range of +/−5 degrees, although a wider range of deflection is also possible. The intraocular OCT device further includes a one-time-use distal portion including a distal cap along with a relay lens and an objective lens, where the relay lens may be inserted into a 25-gauge tube or similar hypotube for insertion into the eye. As shown in FIG. 5A, the proximal end of the relay lens may be brought into close proximity to the MEMS mirror (e.g., within about 0.125 mm) to direct reflected light into the end of the relay lens. Given that the light has been collimated, other distances (e.g., between the collimator and the fixed reflector and between the fixed and moveable reflectors) are flexible.

FIGS. 6A-6C illustrate different cross-sectional three-dimensional renderings of a mechanical design of the intraocular OCT probe 16. FIGS. 6A and 6B show cross-sectional three-dimensional renderings of a mechanical design of the intraocular OCT probe, including a fiber collimator 44, a fixed reflector 48, and a MEMS mirror 46. FIG. 6C shows a cross-sectional three-dimensional rendering of a mechanical design of the intraocular OCT probe, including a strain relief 40, a proximal cap 42, a fiber collimator 44, a fixed reflector 48, and a MEMS mirror 46.

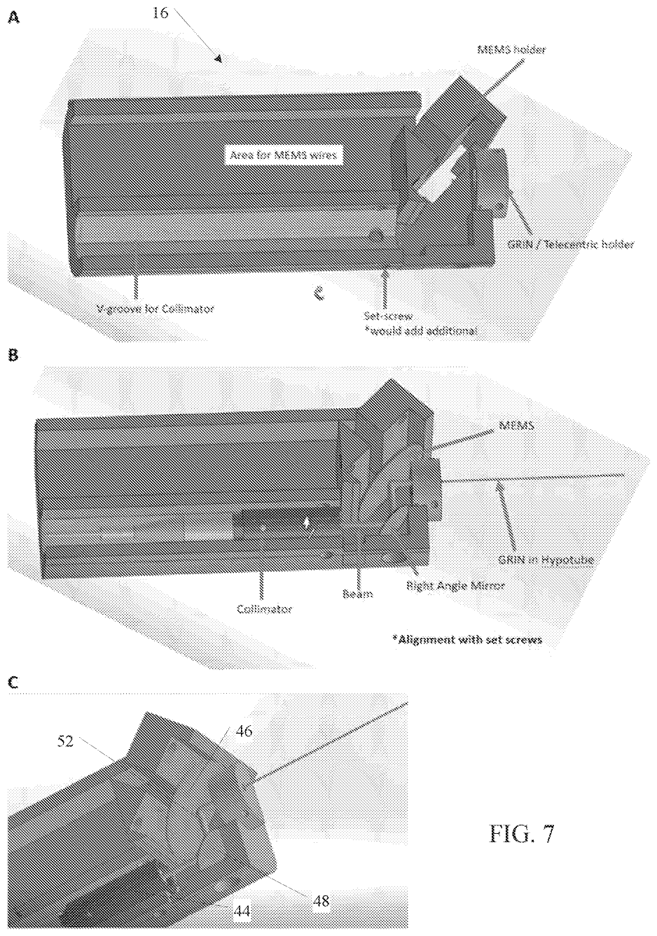

The hand piece may contain various components to assist in an appropriate optical alignment as well as to improve the ease of manufacturing. The hand piece may contain an upper and a lower half in one design. FIGS. 7A-7C show different mechanical models that illustrate the lower half of the body of the OCT probe. The lower half of the hand piece is configured to consist of alignment components for the optics that enable precise alignment and testing prior to the closure of the device. FIG. 7A shows a mechanical model illustrating the lower half of the body of the intraocular OCT probe demonstrating a V-groove for optical alignment, including a V-groove for a collimator, an area for MEMS wire, a MEMS holder, a set screw, and a GRIN/telecentric holder. In the event that the optics are misaligned, in various embodiments adjustment mechanisms such as set screws may be provided in connection with particular components, such as the fiber collimator and the telecentric lens, to enable precise, three-point alignment to achieve a desired alignment and resolution after the components have been assembled. FIG. 7B shows a mechanical model illustrating the lower half of the body of the intraocular OCT probe demonstrating a collimator in place that shines a beam into optics and is placed in a hypotube, including a collimator, a beam, a right angle mirror, a Microelectrical Mechanical System (MEMS), and a GRIN that is in a hypotube. FIG. 7C shows a mechanical model illustrating the lower half of the body of the intraocular OCT probe demonstrating a close-up image of a beam reflecting off of a surface, including a Microelectrical Mechanical System (MEMS), a hypotube, a right angle mirror, a collimator, and a beam.

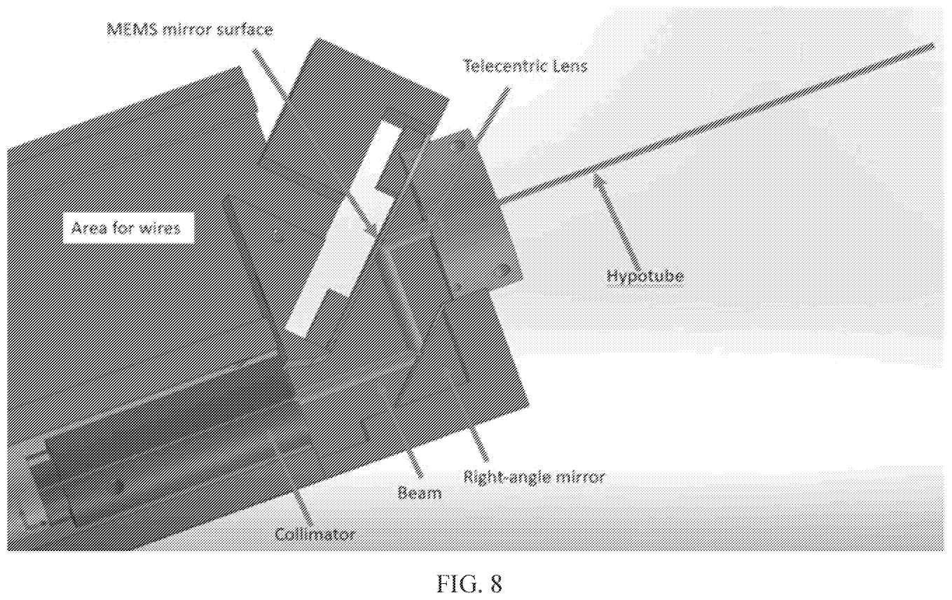

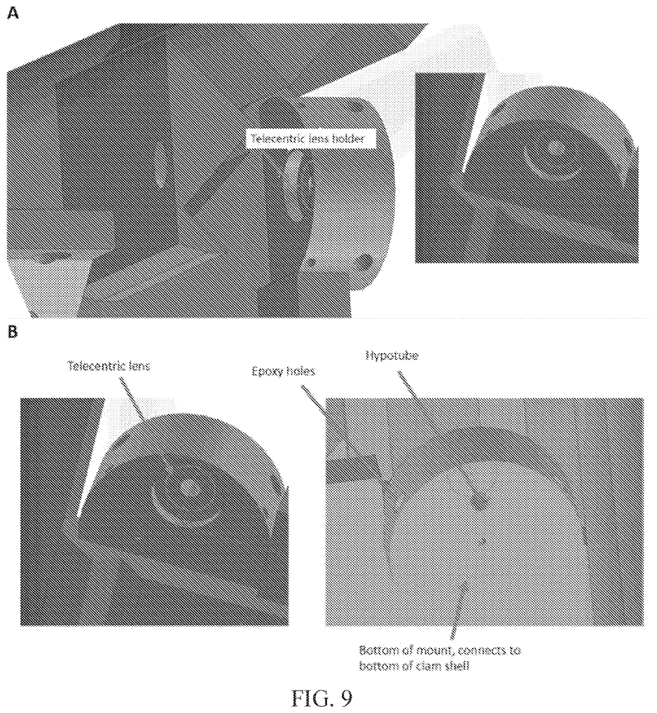

In certain embodiments, the optical design for the one-time use cap may include a telecentric lens to focus the scanning beam onto the proximal end of the relay lens (e.g., a GRIN lens that is part of the relay lens, FIGS. 8 and 9). The relay lens is the only optic component that enters the eye, where it serves to relay and focus the light into the eye at the desired working distance, and thus it is important to properly direct light from the MEMS mirror into the proximal end of the relay lens. FIG. 8 shows a mechanical model demonstrating the alignment of optical components on the interior of the intraocular OCT probe that includes an area for wires, a collimator, a beam, a right angle mirror, and a Microelectrical Mechanical System (MEMS) mirror surface. FIG. 9A shows a mechanical model of a telecentric lens holder with three points of contact for optimal positioning, providing an embodiment of a design for a telecentric lens holder for use with the intraocular OCT probe. FIGS. 9B and 9C show front and rear views of an embodiment of a telecentric lens holder which demonstrate how a hypotube may be coupled to the telecentric lens holder via insertion into an opening on the rear face of the telecentric lens holder.

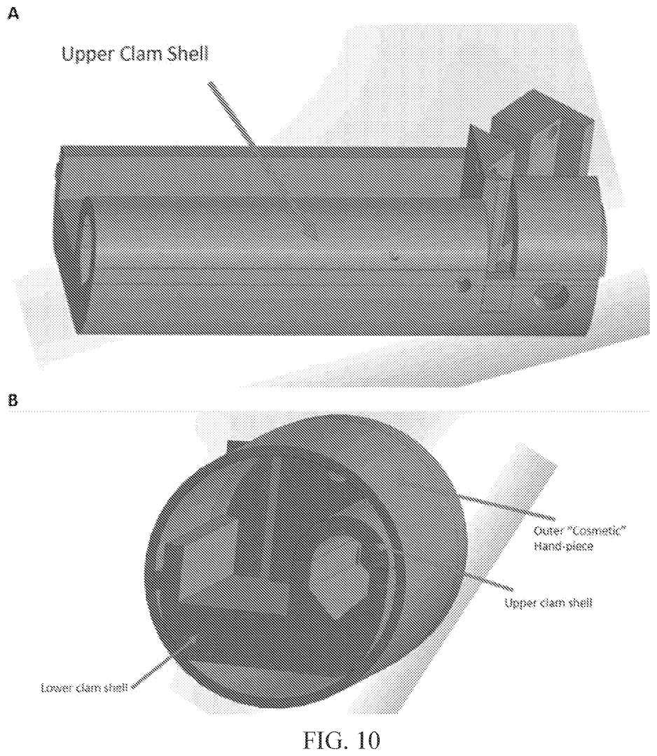

As disclosed above, the hand piece in various embodiments may contain two components, a lower portion or “clam shell” into which optical components are initially placed for alignment, and an upper portion or “clam shell” that closes off the optics, both of which would be enclosed in an outer cosmetic hand piece (FIG. 10). FIG. 10A shows a lateral view of a mechanical model for the upper housing of the body of the intraocular OCT probe that includes an upper clam shell. FIG. 10B shows a distal view of a mechanical model for the upper housing of the body of the intraocular OCT probe, including a lower clam shell, an upper clam shell, and an outer cosmetic hand piece.

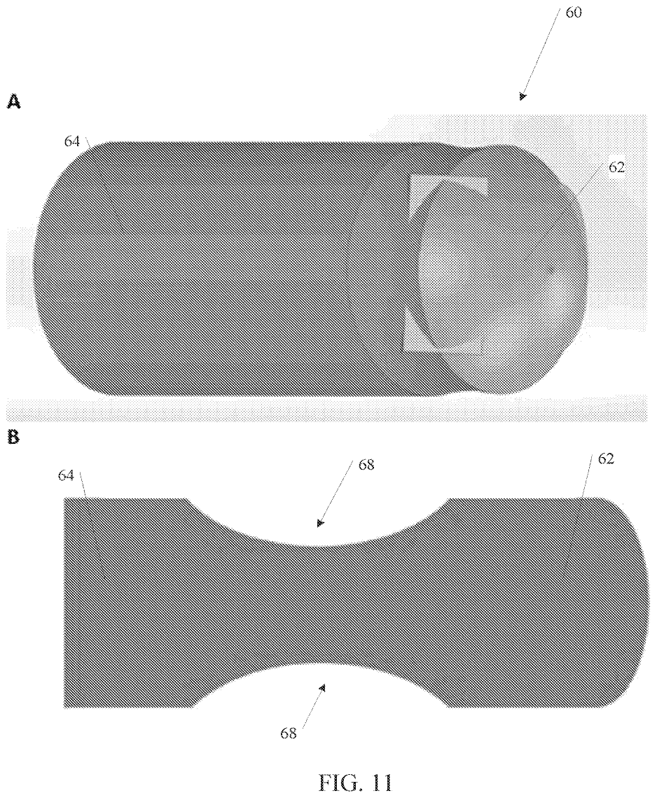

The outer cosmetic hand-piece in various embodiments is designed to have a comfortable ergonomic design and to be similar in dimensions to existing intraocular operative tools as well as sterilizable and safe for re-use in multiple surgical cases (FIGS. 11A, 11B). FIG. 11A shows a mechanical model of an outer design which would transition from hand-piece to a hypotube with a strain relief as a solid tube design of the intraocular OCT probe. FIG. 11B shows a mechanical model of an outer design which would transition from hand-piece to hypotube with a strain relief as ergonomic handles of the intraocular OCT probe.

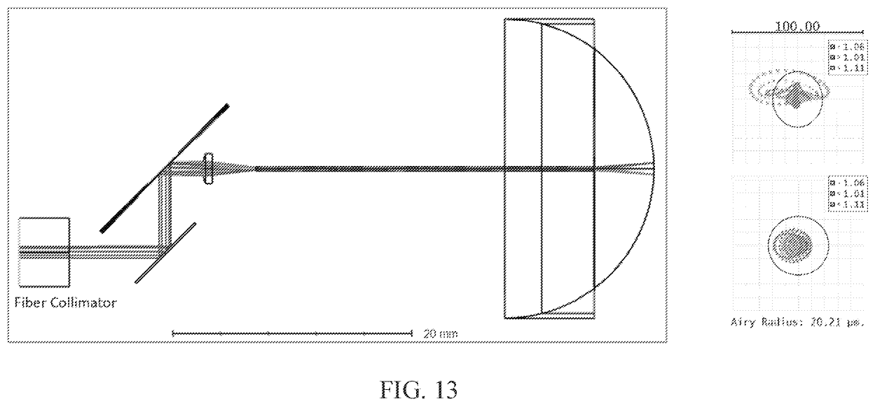

An important aspect of the feasibility of this device is the GRIN lens design which is small enough to fit within a 25-gauge stainless steel tube while still achieving the desired resolution. Extensive optical modeling has been performed to test and validate the optical design (FIGS. 12 and 13).

FIGS. 12 and 13 show optical modeling that has been performed to test and validate the optical design. FIGS. 12A-12C provide output from optical modeling software that tests and validates the optical design of various embodiments of the OCT probe. FIG. 12A shows optical modeling of the entire intraocular OCT probe. FIG. 12B shows optical modeling of the distal tip of the intraocular OCT probe, from the optical fiber (left) to the emitted light applied to the sample (right). FIG. 12C shows optical modeling of an intraocular probe including a relay lens with a plurality of stacked GRIN lenses, where the stack of GRIN lenses includes 12.5 GRIN lenses, the outer diameter is 0.35 millimeters, and the input light may range from 670-1550 nm.

FIG. 13 shows an example of optical modeling which evaluates a spot size and field of view (FOV) of a probe which includes a fiber collimator, a fixed/flat mirror, a Microelectrical Mechanical System (MEMS) and mirror, and relay lens including a GRIN lens.

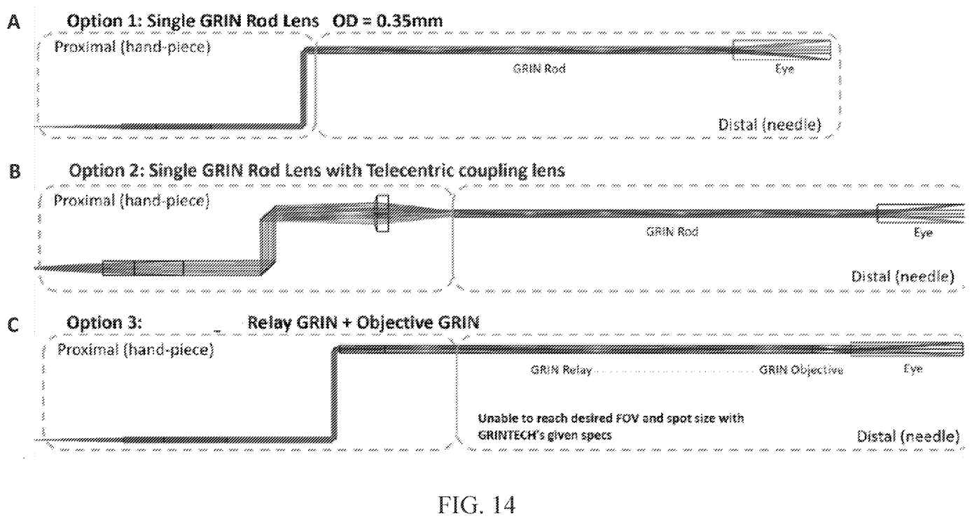

In other designs, a relay lens and an objective lens can be used instead of a long GRIN lens, or the telecentric lens can be removed; these modified designs have also been modeled (FIGS. 14, 15, and 16). FIGS. 14A-14C demonstrate optical modeling of various needle optical designs that can be utilized in the OCT probe, that include different spot sizes and working distances. FIG. 14A shows optical modeling of an embodiment of the probe which includes a single GRIN rod lens with an outer diameter of 0.35 millimeters, where the locations of the proximal (hand-piece), the GRIN rod, the locations of the eye, and the distal (needle) regions are indicated for reference. FIG. 14B shows optical modeling of an embodiment of the probe which includes a single GRIN rod lens with a telecentric coupling lens, where the locations of the proximal (hand-piece), the GRIN rod, the eye, and the distal (needle) regions are indicated for reference. FIG. 14C shows optical modeling of an embodiment of the probe using a design which includes a combination of a relay GRIN lens plus an objective GRIN lens, where the locations of the proximal (hand-piece), the GRIN relay, the GRIN objective, the eye, and the distal (needle) regions are indicated for reference.

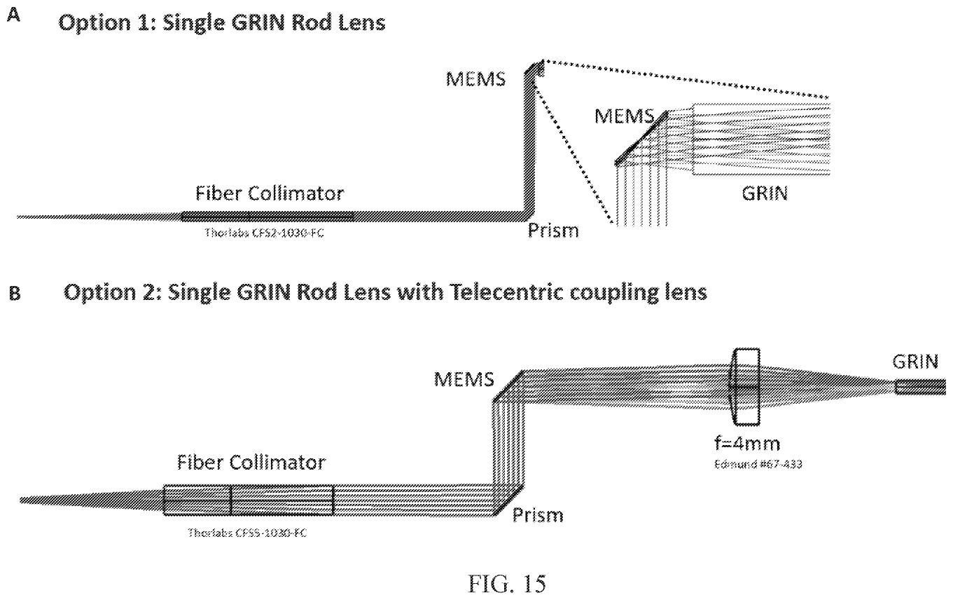

FIGS. 15A and 15B present results of optical modeling of the proximal optical system with and without a telecentric lens. FIG. 15A shows optical modeling of a proximal optical system with the single GRIN rod lens of FIG. 14A, showing the locations of a fiber collimator, a MEMS imaging system, a prism, and a GRIN rod lens for reference. FIG. 15B shows optical modeling of an embodiment of a proximal optical system with a single GRIN rod lens with a telecentric coupling lens as in FIG. 14B, showing the locations of a fiber collimator, a MEMS imaging system, a prism, and a GRIN rod lens for reference, where the telecentric lens has a 4 mm depth of focus.

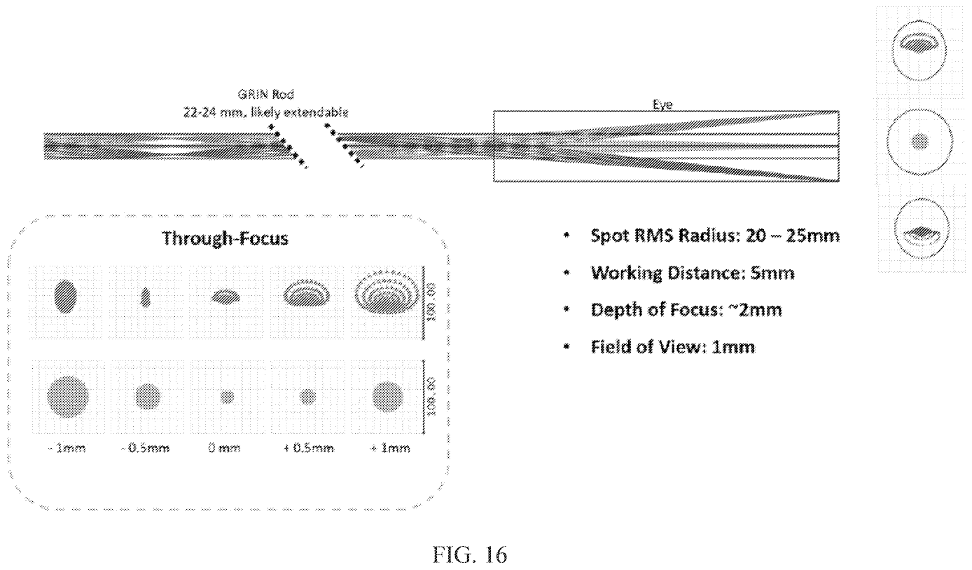

FIG. 16 shows optical modeling including various spot sizes of an intraocular OCT system with a single relay lens and no objective lens, where the locations of the GRIN rod the eye are shown for reference. The inset at the bottom left shows a through-focus series of spots at different focal levels at a point off the central axis (top row) or on the axis (bottom row). The inset at the top right shows the in-focus spots (0 mm depth) at the center and off to the sides of the central optical axis, where the deflections are due to changes in the angle of the moveable reflector (e.g., the MEMS mirror). In this particular optical modeling example, the length of the GRIN rod may be 22-24 millimeters, the spot RMS radius is between 20 and 25 millimeters, the working distance is 5 millimeters, the depth focus may be approximately 2 millimeters, and the field of view is 1 millimeter.

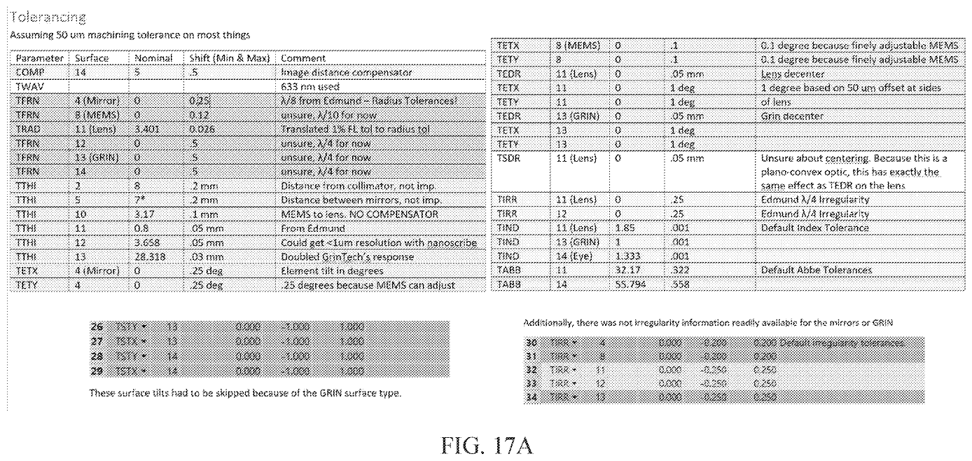

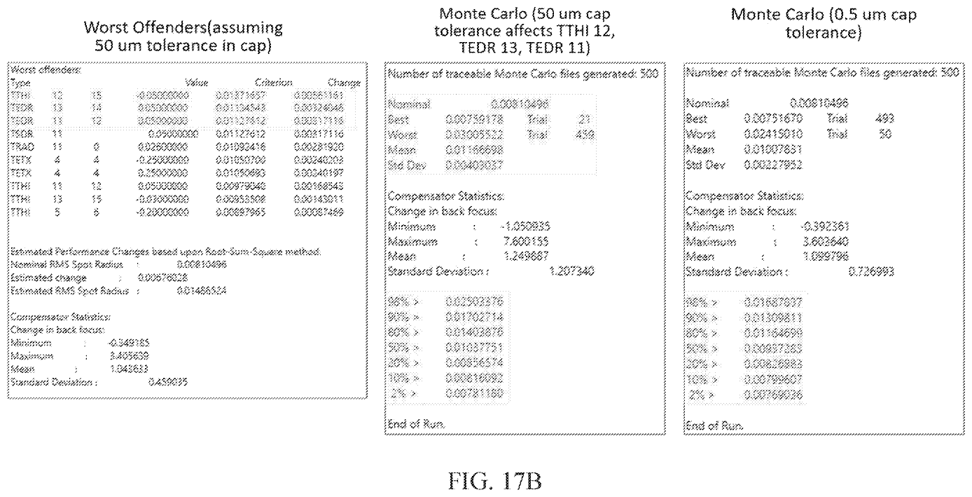

FIG. 17A shows data pertaining to a tolerancing analysis for each device component of the disclosed intraocular OCT probe, including a surface value, nominal value, a shift with a minimum and a maximum value, and a comment, that assure the manufacturing feasibility. FIG. 17B shows further tolerancing analysis data for each device component of the intraocular OCT probe, including a worst offenders' type, value, criterion, and change values, as well as a nominal, best, worst, mean, and standard deviation values, which also help to assure the manufacturing feasibility.

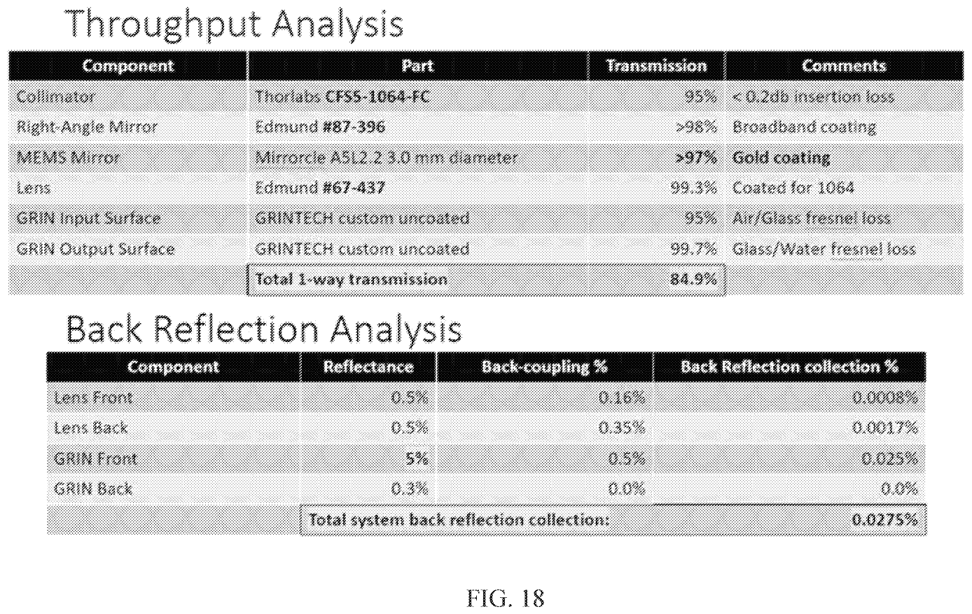

FIG. 18 shows data pertaining to throughput and back reflection analyses for an embodiment of an intraocular OCT probe, indicating that total one-way transmission is almost 85% and total back reflection collection is 0.0275%. The optical design is configured to be optimized to increase the throughput and coupling efficiency back to the optical fiber for the range of wavelengths used in the swept-source OCT. Additionally, the back reflection coupling has been minimized by angle polishing and tilting the GRIN lens. These optimizations increase the signal-to-noise ratio, which is important for obtaining a clean OCT image.

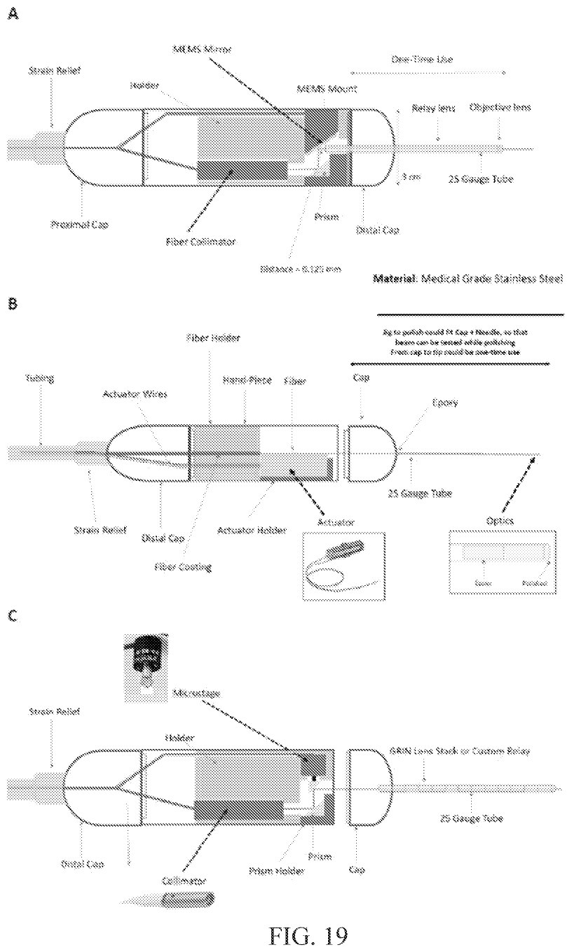

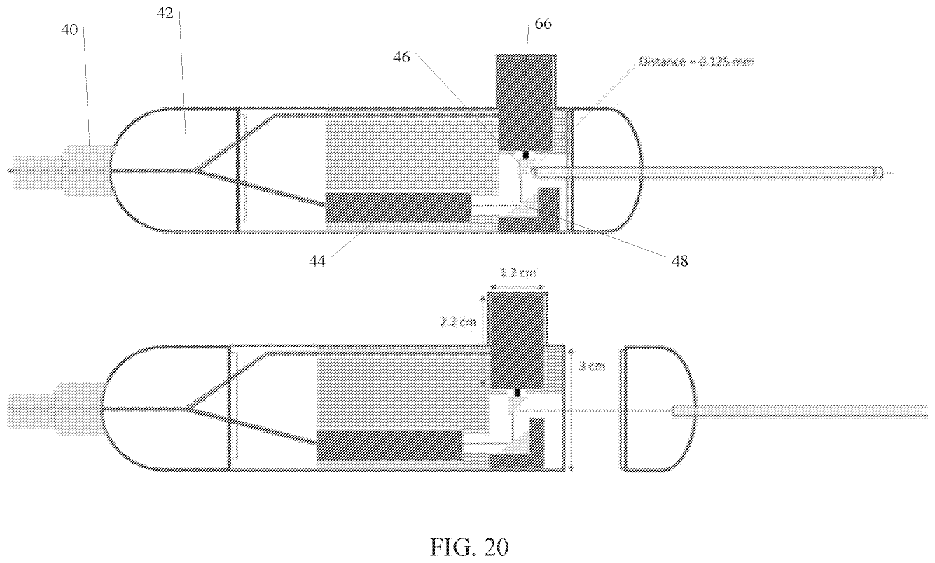

FIGS. 19A-19C and 20 show mechanical models of various embodiments of the probe with different actuators and optical designs have been generated to demonstrate feasibility. FIGS. 19A-19C demonstrate a mechanical illustration of the overall intraocular OCT device design using a MEMS scanning system (FIG. 19A), a single fiber actuator (FIG. 19B), and a rotary motor or galvo (galvanometer) scanning system (FIG. 19C). FIG. 19A shows a mechanical illustration of an intraocular OCT device design which uses a MEMS scanning system, including a strain relief, a proximal cap, a holder, a MEMS mirror, a fiber collimator, a prism, a MEMS mount, and a one-time-use section including a distal cap, a relay lens, an objective lens, and a 25-gauge tube. FIG. 19B shows a mechanical illustration of an intraocular OCT device design which uses a single fiber actuator, including tubing, a strain relief, a distal cap, actuator wires, a fiber holder, a fiber coating, a hand-piece, an amplified actuator, an actuator holder, a fiber, a cap, an epoxy, a 25-gauge tube, and optics.

The actuator expands and contracts to move the fiber. This adjusts where the laser beam enters the distal GRIN optics, which in turn scans the beam at the desired angles. FIG. 19C shows a mechanical illustration of an intraocular OCT device design using a rotary motor or galvo scanning system, including a strain relief, a distal cap, a holder, a collimator, a microstage, a prism, a prism holder, a cap, a GRIN lens stack or custom relay, and a 25-gauge tube.

FIG. 20A shows a mechanical illustration of an embodiment of an intraocular OCT device that accommodates a rotary motor 66 that extends beyond the surface of the housing by modifying the hand-piece design of the of the intraocular OCT probe, where the probe includes a strain relief 40, a proximal cap 42 attached to the body of the OCT probe, a fiber collimator 44, a fixed reflector/prism 48, a moveable reflector/MEMS mirror 46, a distal cap, and a 25-gauge tube/hypotube with a relay lens disposed therein. FIG. 20B shows a mechanical illustration of the intraocular OCT device of FIG. 20A where the distal cap and hypotube/relay lens are shown detached from the main body of the probe.

In addition, beyond the current design there are various modifications that could be made based on the desired surgical applications. In one embodiment, the intraocular OCT probe may be configured to reflect the laser beam at an angle by use of one or more angled polish GRIN lens or prism in the relay lens to direct light along a curved or angled path, where the curved or angled probe may be suitable for imaging the peripheral retina.

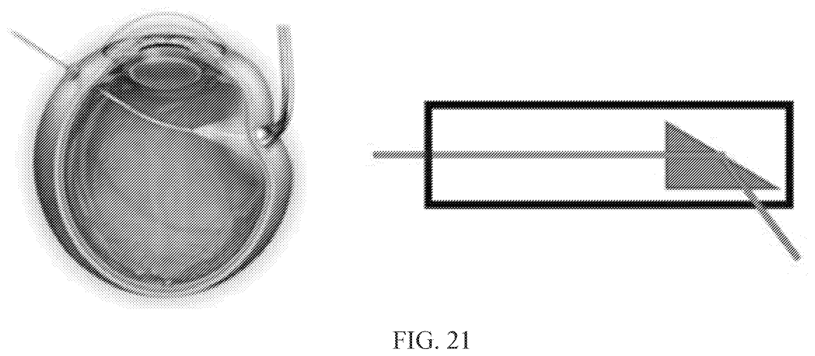

FIG. 21 shows a demonstration of a curved or angled probe design that may be used for imaging the peripheral retina, including an OCT probe.

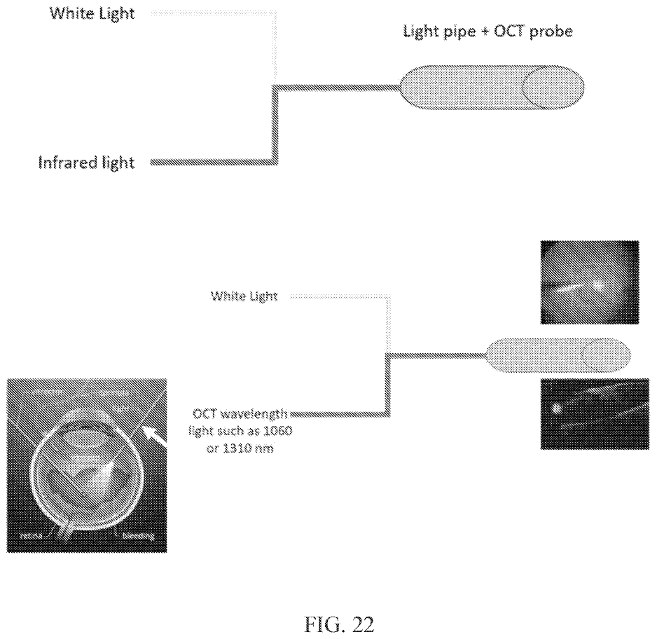

In some embodiments, the intraocular OCT probe is configured to perform both white light and OCT imaging through the same probe. This may be achieved by adding a splitter at a proximal end of the probe (e.g., proximal to the strain relief) to connect the optical fiber to both a white light source and a laser source. FIG. 22, top panel, shows a diagram of a probe design for performing both white light imaging and OCT imaging simultaneously, including as shown diagrammatically in the bottom panel, a white light image and an infrared light/OCT image. The output image may be presented by projecting both the white light and OCT images onto the monitor in a split-screen/side-by-side image view.

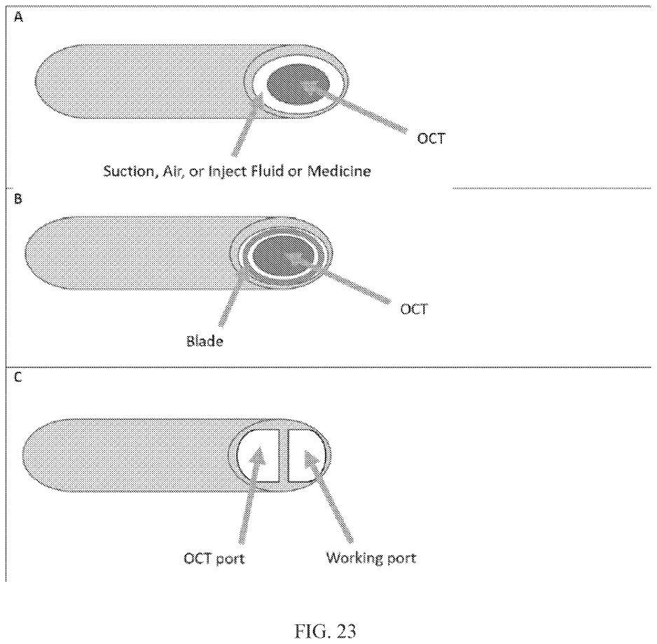

FIGS. 23A-23C demonstrate various embodiments of functions that may be performed along with OCT imaging using the intraocular probe. FIG. 23A shows a design of a multi-channel intraocular OCT probe with an OCT lens or fiber in the center channel and one or more of suction, air, injected fluid, or medicament being applied to one or more outer channels. FIG. 23B shows a design of a multi-channel intraocular OCT probe with an OCT lens or fiber in the center channel and a biopsy or cutting blade disposed in one or more outer channels, where the biopsy or cutting blade may be concentric with the OCT probe. FIG. 23C shows a design of a multi-channel intraocular OCT probe with a septum between the channels and an OCT lens or fiber in one of the channels and a second channel being available as a working port. Among other functions, the working port may be used to perform suctioning or injection during the procedure.

Compared to prior intraocular OCT devices, the disclosed intraocular OCT probe provides certain advantages. In some embodiments, these design aspects allow the device to be manufactured at a large scale and adopted more widely. Previous intraocular OCT devices were predicated on the utility of a single-mode fiber in the needle tip, which posed risks for long term durability and image stability. The disclosed design, on the other hand, uses a fiber collimator placed in the hand-piece as opposed to the needle to improve image quality and durability. The disclosed collimator-based design, compared to the single-mode fiber designs, is also easier to manufacture, more durable, and allows for incorporation of a one-time use tip. The removable/one-time use tip is important for clinical feasibility from a contamination and infection standpoint, as well as the fact that miniature optical pieces are likely to be damaged if used multiple times. In addition, the actuator in the disclosed device is much less complicated compared to prior actuator designs and can be purchased off-the-shelf, instead of requiring custom and complicated machining. Overall, the disclosed design is more cost-effective, more clinically feasible, and more likely to scale into a device that can be used nationally and globally in vitreoretinal surgery, and potentially other fields of ophthalmic surgery. Overall, there are numerous embodiments captured in designs and iterations that are considered and have been listed above and represented in the figures.

OCT continues to be an important technology in the field of ophthalmology, with further technological advances and machine learning-based applications influencing understanding of ophthalmic diseases and improving patient outcomes. The disclosed intraocular OCT device will allow for further advances in the field of intraoperative OCT for vitreoretinal and other ophthalmic surgeries.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. An optical probe, comprising:
an optical fiber coupled to a moveable actuator,
the moveable actuator being configured to direct light emitted from the optical fiber at a plurality of varying angles; and
a relay lens distal to the moveable actuator and configured to receive light emitted from the optical fiber and direct the received light at a sample.

2. The optical probe of claim 1, further comprising:
a fiber collimator disposed at a distal end of the optical fiber;
a fixed reflective surface disposed distal to the fiber collimator; and
an adjustable reflective surface coupled to the moveable actuator and disposed adjacent the fixed reflective surface,
wherein the adjustable reflective surface is configured to reflect light emitted from the fiber collimator and reflected off the fixed reflective surface at the plurality of varying angles, and
wherein the relay lens is configured to receive light reflected from the adjustable reflective surface and direct the received light at the sample.

3. The optical probe of claim 2, wherein the moveable actuator comprises at least one of a Microelectrical Mechanical System (MEMS) device, a galvanometer, or a rotary motor.

4. The optical probe of any one of claims 1-3, wherein the relay lens is disposed within a distal cap, and
wherein the distal cap and the relay lens are detachable from the optical probe.

5. The optical probe of any one of claims 1-4, wherein the relay lens comprises an objective lens at a distal end thereof.

6. The optical probe of claim 1, wherein the relay lens comprises a GRIN lens.

7. The optical probe of claim 6, wherein the GRIN lens comprises a plurality of stacked GRIN lenses.

8. The optical probe of claim 7, wherein the relay lens is a curved or angled probe, and
wherein the relay lens comprises at least one of an angle-polished GRIN lens or a prism that is configured to direct light through the curved or angled probe.

9. The optical probe of any one of claims 1-8, further comprising a telecentric lens disposed between the moveable actuator and the relay lens,
wherein the telecentric lens is configured to focus the light emitted from the optical fiber onto a proximal end of the relay lens.

10. The optical probe of any one of claims 1-9, wherein the moveable actuator rotates on a first axis such that the received light is scanned in a first direction across a surface of the sample.

11. The optical probe of claim 10, wherein the moveable actuator rotates on a second axis perpendicular to the first axis such that the received light is scanned in a second direction perpendicular to the first direction across the surface of the sample.

12. The optical probe of claim 2, wherein the fixed reflective surface comprises at least one of a mirror or a prism.

13. The optical probe of claim 1, wherein the optical fiber and the moveable actuator are disposed within a hand piece.

14. The optical probe of claim 13, wherein the hand piece comprises a lower portion and an upper portion, and
wherein the optical fiber and the moveable actuator are disposed in the lower portion of the hand piece to facilitate optical alignment, and
wherein the upper portion of the hand piece is secured to the lower portion of the hand piece.

15. The optical probe of claim 14, wherein the hand piece further comprises a plurality of adjustment mechanisms associated with at least one of the optical fiber and the moveable actuator, and
wherein the plurality of adjustment mechanisms are configured to facilitate the optical alignment.

16. The optical probe of claim 15, wherein the hand piece is disposed within an outer housing.

17. The optical probe of claim 2, wherein the fiber collimator, the fixed reflective surface, the moveable actuator, and the adjustable reflective surface are disposed within a hand piece.

18. The optical probe of claim 17, wherein the hand piece comprises a lower portion and an upper portion, and
wherein the fiber collimator, the fixed reflective surface, the moveable actuator, and the adjustable reflective surface are disposed in the lower portion of the hand piece to facilitate optical alignment, and
wherein the upper portion of the hand piece is secured to the lower portion of the hand piece.

19. The optical probe of claim 18, wherein the hand piece further comprises a plurality of adjustment mechanisms associated with at least one of the fiber collimator, the fixed reflective surface, the moveable actuator, and the adjustable reflective surface, and
wherein the plurality of adjustment mechanisms are configured to facilitate the optical alignment.

20. The optical probe of claim 19, wherein the hand piece is disposed within an outer housing.

21. The optical probe of any one of claims 1-20, wherein the plurality of varying angles comprises angles of up to 3.75 degrees on either side of an optical axis.

22. The optical probe of any one of claims 1-21, wherein the relay lens is disposed within a hypotube.

23. The optical probe of claim 22, wherein the hypotube comprises a plurality of channels, and
wherein the relay lens is disposed within a first channel of the plurality of channels.

24. The optical probe of claim 23, wherein the plurality of channels comprises at least two channels separated by a septum.

25. The optical probe of claim 23, wherein the plurality of channels comprises a plurality of concentric channels,
wherein the relay lens is disposed within a first central channel.

26. The optical probe of claim 25, wherein a second outer channel comprises at least one of a cutting blade, a suction port, an air delivery port, a fluid delivery port, or a medicament delivery port.

27. The optical probe of any one of claims 1-26, further comprising an optical coherence tomography (OCT) system coupled to the optical fiber.

28. The optical probe of claim 27, wherein the OCT system comprises an OCT light source coupled to the optical fiber.

29. The optical probe of claim 28, further comprising a white light source coupled to the optical fiber.

30. The optical probe of any one of claims 1-29, wherein the sample comprises a retina.

31. A method of operating an optical probe, comprising:
providing an optical fiber coupled to a moveable actuator;
directing light emitted from the optical fiber at a plurality of varying angles;
receiving, by a relay lens distal to the moveable actuator, light emitted from the optical fiber; and
directing the received light at a sample.

32. The method of claim 31, further comprising:
providing:
a fiber collimator disposed at a distal end of the optical fiber,
a fixed reflective surface disposed distal to the fiber collimator, and
an adjustable reflective surface coupled to the moveable actuator and disposed adjacent the fixed reflective surface;
reflecting, using the adjustable reflective surface, light emitted from the fiber collimator and reflected off the fixed reflective surface at the plurality of varying angles;
receiving, by the relay lens, light reflected from the adjustable reflective surface; and
directing, by the relay lens, the received light at the sample.

33. The method of claim 32, wherein the moveable actuator comprises at least one of a Microelectrical Mechanical System (MEMS) device, a galvanometer, or a rotary motor.

34. The method of any one of claims 31-33, wherein the relay lens is disposed within a distal cap, and
wherein the method further comprises:
detaching the distal cap and the relay lens from the optical probe.

35. The method of any one of claims 31-34, wherein the relay lens comprises an objective lens at a distal end thereof.

36. The method of claim 31, wherein the relay lens comprises a GRIN lens.

37. The method of claim 36, wherein the GRIN lens comprises a plurality of stacked GRIN lenses.

38. The method of claim 37, wherein the relay lens is a curved or angled probe,
wherein the relay lens comprises at least one of an angle-polished GRIN lens or a prism, and
wherein the method further comprises:
directing light through the curved or angled probe via the at least one of an angle-polished GRIN lens or prism.

39. The method of any one of claims 31-38, further comprising a telecentric lens disposed between the moveable actuator and the relay lens, and
wherein the method further comprises:
focusing, using the telecentric lens, the light emitted from the optical fiber onto a proximal end of the relay lens.

40. The method of any one of claims 31-39, further comprising:
rotating the moveable actuator on a first axis such that the received light is scanned in a first direction across a surface of the sample.

41. The method of claim 40, further comprising:
rotating the moveable actuator on a second axis perpendicular to the first axis such that the received light is scanned in a second direction perpendicular to the first direction across the surface of the sample.

42. The method of claim 32, wherein the fixed reflective surface comprises at least one of a mirror or a prism.

43. The method of claim 31, wherein the optical fiber and the moveable actuator are disposed within a hand piece.

44. The method of claim 43, wherein the hand piece comprises a lower portion and an upper portion, and
wherein the method further comprises:
disposing the optical fiber and the moveable actuator in the lower portion of the hand piece to facilitate optical alignment, and
securing the upper portion of the hand piece to the lower portion of the hand piece.

45. The method of claim 44, wherein the hand piece further comprises a plurality of adjustment mechanisms associated with at least one of the optical fiber and the moveable actuator, and
performing optical alignment using the plurality of adjustment mechanisms.

46. The method of claim 45, further comprising:
disposing the hand piece within an outer housing.

47. The method of claim 32, further comprising:
disposing the fiber collimator, the fixed reflective surface, the moveable actuator, and the adjustable reflective surface within a hand piece.

48. The method of claim 47, wherein the hand piece comprises a lower portion and an upper portion, and
wherein the method further comprises:
disposing the fiber collimator, the fixed reflective surface, the moveable actuator, and the adjustable reflective surface in the lower portion of the hand piece to facilitate optical alignment, and
securing the upper portion of the hand piece to the lower portion of the hand piece.

49. The method of claim 48, wherein the hand piece further comprises a plurality of adjustment mechanisms associated with at least one of the fiber collimator, the fixed reflective surface, the moveable actuator, and the adjustable reflective surface, and
wherein the method further comprises:
performing the optical alignment using the plurality of adjustment mechanisms.

50. The method of claim 49, further comprising:
disposing the hand piece within an outer housing.

51. The method of any one of claims 31-50, wherein directing light emitted from the optical fiber at a plurality of varying angles further comprises:
directing light emitted from the optical fiber at a plurality of varying angles,
wherein the plurality of varying angles comprises angles of up to 3.75 degrees on either side of an optical axis.

52. The method of any one of claims 31-51, further comprising:
disposing the relay lens within a hypotube.

53. The method of claim 52, wherein the hypotube comprises a plurality of channels, and
wherein the method further comprises:
disposing the relay lens within a first channel of the plurality of channels.

54. The method of claim 53, wherein the plurality of channels comprises at least two channels separated by a septum.

55. The method of claim 53, wherein the plurality of channels comprises a plurality of concentric channels, and
wherein the method further comprises:
disposing the relay lens within a first central channel.

56. The method of claim 55, wherein a second outer channel comprises at least one of a cutting blade, a suction port, an air delivery port, a fluid delivery port, or a medicament delivery port.

57. The method of any one of claims 31-56, further comprising an optical coherence tomography (OCT) system coupled to the optical fiber.

58. The method of claim 57, wherein the OCT system comprises an OCT light source coupled to the optical fiber.

59. The method of claim 58, further comprising:
coupling a white light source the optical fiber.

60. The method of any one of claims 31-59, wherein the sample comprises a retina.
